(19) United States
(12) Patent Application Publication
(10) Pub. No.: US 2023/0122523 A1
(43) Pub. Date: Apr. 20, 2023

(54) HYDROGEN SENSOR ELEMENT

(71) Applicant: SUMITOMO CHEMICAL COMPANY, LIMITED, Tokyo (JP)

(72) Inventors: Megumi HAYASAKA, Osaka-shi (JP); Yuichiro KUNAI, Arlington, MA (US)

(73) Assignee: SUMITOMO CHEMICAL COMPANY, LIMITED, Tokyo (JP)

(21) Appl. No.: 17/911,617

(22) PCT Filed: Feb. 3, 2021
(86) PCT No.: PCT/JP2021/003879

§ 371 Date: Sep. 14, 2022

(30) Foreign Application Priority Data
Mar. 26, 2020 (JP) 2020-055638
Oct. 14, 2020 (JP) 2020-173094

(51) Int. Cl. G01N 33/00; G01N 27/12

(52) U.S. Cl. CPC G01N 33/005; G01N 27/127; G01N 27/126

(57) ABSTRACT

A hydrogen sensor element comprising a pair of electrodes and a hydrogen detection film disposed in contact with the pair of electrodes, wherein the hydrogen detection film contains a conjugated polymer and a dopant, and wherein the absolute value |ΔG| of energy difference between the lowest unoccupied orbital of the dopant and the highest occupied orbital of the conjugated polymer in the ground state is 4.5 eV or more, is provided.

### TECHNICAL FIELD

The present invention relates to a hydrogen sensor element.

### BACKGROUND ART

As conventional hydrogen sensor elements, a contact combustion type and a semiconductor type are mainly known.

The contact combustion type hydrogen sensor element uses a noble metal such as platinum and palladium as combustion catalyst and tin oxide or alumina as support material in a detection unit, and hydrogen is detected by detecting increase in the element temperature due to combustion of hydrogen through catalytic reaction.

The semiconductor type hydrogen sensor element uses a platinum wire coil coated with fine particles of indium oxide or the like as detection unit. When an oxidation reaction of hydrogen occurs in the detection unit, negative ionized oxygen adsorbed on the surface of the fine particles is consumed. As a result, free electrons are generated to reduce the electric resistance value. The semiconductor type hydrogen sensor element detects hydrogen by detecting the decrease in the electric resistance value.

In any of the contact combustion type and the semiconductor type, the detection unit is required to be heated to several hundred degrees or more, so that the power consumption is large and there is room for improvement in safety. Further, since an inorganic sintered body is used in any of the methods, it is usually difficult to impart flexibility to the hydrogen sensor element.

In Non patent Literature 1, a hydrogen sensor element equipped with a hydrogen detection film made of a composite including polyaniline and TiO₂ doped with camphorsulfonic acid is disclosed.

### CITATION LIST

### Non Patent Literature

Non Patent Literature 1

Subodh Srivastava, Sumit Kumar, V. N. Singh, M. Singh, Y. K. Vijay, Synthesis and characterization of TiO2doped polyaniline composites for hydrogen gas sensing, International Journal of Hydrogen Energy 36 (2011) 6343-6355

### SUMMARY OF INVENTION

### Technical Problem

It is preferable that a hydrogen sensor element that detects hydrogen based on the increase or decrease in the electric resistance value have good sensitivity to changes in the hydrogen concentration of a measurement target, from the viewpoint of enhancing the function and/or reliability as a sensor. The sensitivity refers to a percentage change in electric resistance value indicated by the hydrogen sensor element. The hydrogen sensor element disclosed in Non Patent Literature 1 has room for improvement in sensitivity.

An object of the present invention is to provide a hydrogen sensor element provided with a hydrogen detection film containing an organic substance, having good sensitivity.

### Solution to Problem

The present invention provides a hydrogen sensor element shown as follows.

[1] A hydrogen sensor element comprising a pair of electrodes and a hydrogen detection film disposed in contact with the pair of electrodes,

the hydrogen detection film containing a conjugated polymer and a dopant, and

the absolute value |ΔG| of energy difference between the lowest empty orbital of the dopant and the highest occupied orbital of the conjugated polymer in the ground state being 4.5 eV or more.

[2] The hydrogen sensor element according to item [1], wherein the conjugated polymer is a polyaniline-based polymer.

[3] The hydrogen sensor element according to item [1] or [2], wherein the dopant is an organic dopant.

[4] The hydrogen sensor element according to any one of items [1] to [3], wherein the hydrogen detection film contains nanofibers of the conjugated polymer.

### Advantageous Effects of Invention

A hydrogen sensor element provided with a hydrogen detection film containing an organic substance, having good sensitivity, can be provided.

### BRIEF DESCRIPTION OF DRAWINGS

FIG. 1 is a schematic top view showing an example of the hydrogen sensor element according to the present invention.

FIG. 2 is a schematic top view showing a method for manufacturing a hydrogen sensor element.

FIG. 3 is a schematic top view showing a method for manufacturing a hydrogen sensor element.

FIG. 4 is a schematic diagram showing a measurement system structure for evaluating the sensitivity of a hydrogen sensor element.

### DESCRIPTION OF EMBODIMENT

A hydrogen sensor element according to the present invention (hereinafter, also simply referred to as “hydrogen sensor element”) comprises a pair of electrodes and a hydrogen detection film disposed in contact with the pair of electrodes.

The hydrogen detection film may be in contact with each of the pair of electrodes. Preferably, the pair of electrodes are disposed oppositely and separated from each other. The hydrogen detection film is disposed in contact with each of the electrodes between the pair of electrodes disposed oppositely.

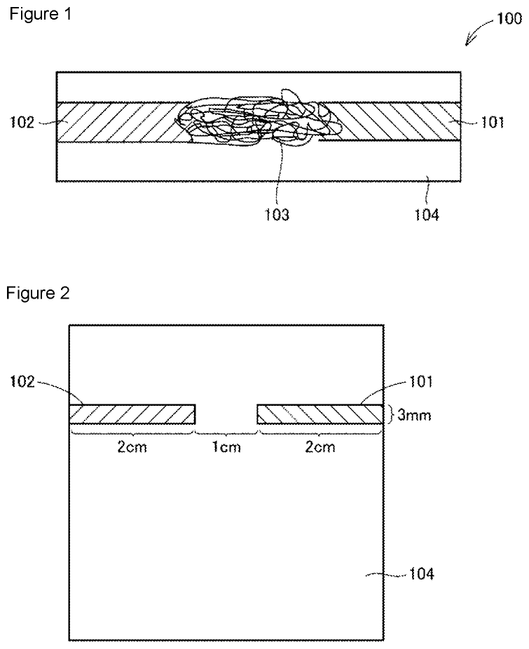

FIG. 1 is a schematic top view showing an example of the hydrogen sensor element. A hydrogen sensor element 100 shown in FIG. 1 comprises a pair of electrodes composed of a first electrode 101 and a second electrode 102, and a hydrogen detection film 103 disposed in contact with both of the first electrode 101 and the second electrode 102. Both ends of the hydrogen detection film 103 are formed on the first electrode 101 and the second electrode 102, respectively, so that the hydrogen detection film 103 is in contact with these electrodes.

The hydrogen sensor element optionally further includes a substrate 104 that supports the first electrode 101, the second electrode 102, and the hydrogen detection film 103 (refer to FIG. 1).

The hydrogen sensor element 100 shown in FIG. 1 detects hydrogen by detecting the decrease/increase in the electric resistance value caused by hydrogen doping/dedoping of a conjugated polymer contained in the hydrogen detection film 103.

[1] First Electrode and Second Electrode

As the first electrode 101 and the second electrode 102, those having a sufficiently smaller electric resistance value than the hydrogen detection film 103 are used. Specifically, the electric resistance values of the first electrode 101 and the second electrode 102 included in the hydrogen sensor element are preferably 500Ω or less, more preferably 200Ω or less, and still more preferably 100Ω or less at a temperature of 25° C.

The materials of the first electrode 101 and the second electrode 102 are not particularly limited as long as an electric resistance value sufficiently smaller than that of the hydrogen detection film 103 can be obtained, and for example, a single metal such as gold, silver, copper, platinum, or palladium; an alloy containing two or more types of metal materials; a metal oxide such as indium tin oxide (ITO) and indium zinc oxide (IZO); and a conductive organic substance (conductive polymer or the like) may be used.

The material of the first electrode 101 and the material of the second electrode 102 may be the same or different.

The method for forming the first electrode 101 and the second electrode 102 is not particularly limited, and may be a generalized method such as vapor deposition, sputtering, or coating (application). The first electrode 101 and the second electrode 102 may be formed directly on the substrate 104.

The thickness of the first electrode 101 and the second electrode 102 is not particularly limited as long as an electric resistance value sufficiently smaller than that of the hydrogen detection film 103 can be obtained, being, for example, 50 nm or more and 1000 nm or less, preferably 100 nm or more and 500 nm or less.

[2] Substrate

The substrate 104 is a support for supporting the first electrode 101, the second electrode 102, and the hydrogen detection film 103.

The material of the substrate 104 is not particularly limited as long as it is non-conductive (insulating), and may be a resin material such as thermoplastic resin and an inorganic material such as glass. With use of a resin material as the substrate 104, since the hydrogen detection film 103 typically has flexibility, the hydrogen sensor element can be imparted with flexibility.

The thickness of the substrate 104 is preferably set in consideration of the flexibility and durability of the hydrogen sensor element. The thickness of the substrate 104 is, for example, 10 μm or more and 5000 μm or less, preferably 50 μm or more and 1000 μm or less.

[3] Hydrogen Detection Film

The hydrogen detection film 103 contains a conjugated polymer and a dopant, and preferably contains a conjugated polymer doped with a dopant. The hydrogen detection film 103 is preferably made of a conjugated polymer and a dopant, and more preferably made of a conjugated polymer doped with a dopant.

It is preferable that the hydrogen detection film 103 have a shape having a large surface area, from the viewpoint of increasing the reactivity with hydrogen gas for improvement in the sensitivity.

Examples of the hydrogen detection film having the above shape include a film composed of nanofibers of a conjugated polymer, with the nanofibers being doped (adsorbed) with a dopant; a film composed of fine particles of a conjugated polymer, with the fine particles being doped (adsorbed) with a dopant; and a film containing a porous material, with the porous material being impregnated with a conjugated polymer and a dopant.

The hydrogen detection film 103 is preferably a film containing nanofibers of a conjugated polymer, with the nanofibers being doped (adsorbed) with an organic dopant, and more preferably a film composed of nanofibers of a conjugated polymer and an organic dopant doped (adsorbed) to the nanofibers.

It is preferable that the hydrogen detection film 103 allow the conjugated polymer to be exposed to the surface, from the viewpoint of enabling contact between the conjugated polymer and hydrogen, preferably from the viewpoint of enabling contact between the conjugated polymer and hydrogen on an surface area as large as possible.

[3-1] Conjugated Polymer

A conjugated polymer usually has an extremely low electrical conductivity of its own, for example, 1×10⁻⁶ S/m or less, exhibiting almost no electrical conduction properties. The electrical conductivity of a conjugated polymer itself is low, because electrons cannot move freely due to saturation of electrons in the valence band. On the other hand, due to delocalization of electrons, a conjugated polymer has a significantly smaller ionization potential and a very large electron affinity in comparison with a saturated polymer. Accordingly, charge transfer tends to be caused between the conjugated polymer and a suitable dopant, for example, an electron acceptor or an electron donor, so that the dopant can pull out an electron from a valence band of the conjugated polymer, or the dopant can inject an electron into a conduction band. Therefore, in a conjugated polymer doped with a dopant, a small number of holes are present in the valence band or a small number of electrons are present in the conduction band, and these can move freely, so that the conductivity tends to be drastically improved.

The conjugated polymer has a value of the single wire resistance R for a distance between lead rods set to several mm to several cm in measurement with an electric tester of preferably in the range of 0.01Ω or more and 300 MΩ or less at a temperature of 25° C. Such a conjugated polymer has a conjugated system structure in the molecule, and examples thereof include a molecule having a skeleton in which double bonds and single bonds are alternately connected, and a polymer having a conjugated unshared electron pair. As described above, such a conjugated polymer can be easily imparted with electrical conduction properties by doping. The conjugated polymer is not particularly limited, and examples thereof include polyacetylene; poly(p-phenylene vinylene); polypyrrole; polythiophene-based polymers such as poly(3,4-ethylenedioxythiophene) [PEDOT]; and polyaniline-based polymers. The polythiophene-based polymers refer to polythiophene, a polymer having a polythiophene skeleton, with a substituent introduced into a side chain, a polythiophene derivative, etc. In the present specification, the term “-based polymer” means a similar molecule.

Only one type of conjugated polymer may be used, or two or more types may be used in combination.

From the viewpoint of easiness in polymerization and identification, it is preferable that the conjugated polymer be a polyaniline-based polymer.

[3-2] Dopant

Examples of the organic dopant include an organic compound that functions as an electron acceptor for a conjugated polymer.

In general, a conjugated polymer is provided with conductive properties through loss of an electron pulled out by a dopant that functions as an electron acceptor.

The doping/dedoping behavior of the dopant for the conjugated polymer is a reversible redox reaction. The doped state is an oxidized state and the chemical potential thereof is high. The conjugated polymer in the doped state acts as an oxidant, and the potential thereof differs depending on the type of the conjugated polymer.

Further, the doping percentage of the dopant for the conjugated polymer varies, and the chemical potential increases as the doping percentage increases. With an excessively high doping percentage, oxidative decomposition of the conjugated polymer itself occurs. The upper limit of the doping percentage that causes no oxidative decomposition differs depending on the type of conjugated polymer.

Using a polyaniline doped with a dopant H⁺A⁻ that functions as an electron acceptor as example, the mechanism how the hydrogen detection film containing a conjugated polymer and a dopant detects hydrogen is described based on the following equation. Incidentally, polyaniline is conductive only in an emeraldine salt state.

A polyaniline doped with a dopant H⁺A⁻ further dopes hydrogen when exposed to hydrogen gas, and as a result, the electric resistance value decreases. Hydrogen gas can be detected by detecting such a fluctuation in the electric resistance value.

The doped hydrogen molecule acts on a nitrogen atom having a positive charge of two polyaniline molecules (the following formulas (a) and (b)). Subsequently, when an N—H bond is formed between the hydrogen molecule and the two polyaniline molecules, the H—H bond in the hydrogen molecule is dissociated (the following formula (c)). Then, in each of the two polyaniline molecules, an electron and A⁻ move between adjacent N atoms (the following formulas (c) and (d)), and on this occasion, the hydrogen atom dissociate from the N atom to form a hydrogen molecule (the following formula (e)).

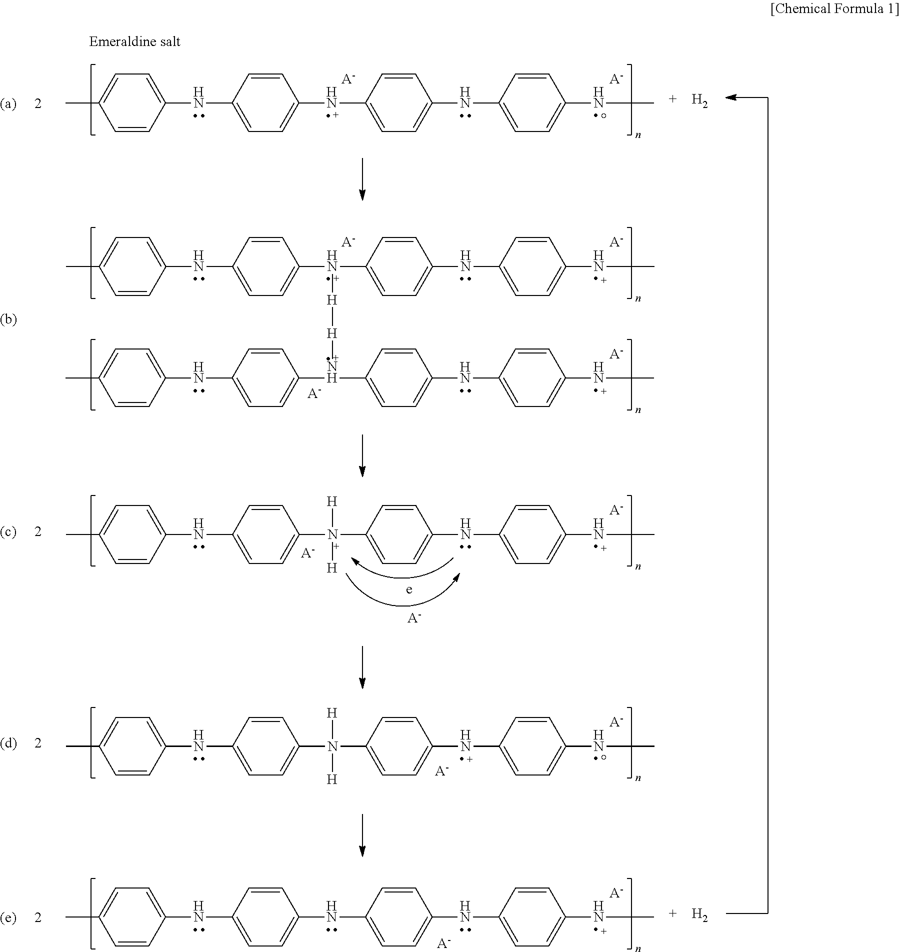

As shown by the mechanism described above, the hydrogen detection film can reversibly react with hydrogen gas, and thereby the hydrogen sensor element can exhibit reversibility of the electric resistance value.

Further, since the hydrogen sensor element provided with the hydrogen detection film containing the conjugated polymer and the dopant detects hydrogen based on the above mechanism, it can be driven at room temperature.

The dopant contained in the hydrogen detection film 103 is selected to have an absolute value |ΔG| of the energy difference between the lowest unoccupied orbital (LUMO) of the dopant and the highest occupied orbital (HOMO) of the conjugated polymer in the ground state of 4.5 eV or more. Thereby, the sensitivity of the hydrogen sensor element can be improved. |ΔG| is represented by the following equation.

|ΔG|=|(LUMO energy of dopant)−(HOMO energy of conjugated polymer)|

Hereinafter, the dopant that satisfies the above formula is also referred to as “dopant (A)”.

The dopant contained in the hydrogen detection film 103 may contain only one type of dopant (A), or may contain two or more types.

In order to enhance the sensitivity (reactivity to hydrogen) of the hydrogen sensor element, it is important to reduce the positive charge of the conjugated polymer. Reducing the positive charge in the conjugated polymer means reducing the attraction of an electron from the conjugated polymer by the dopant, which leaves room for attraction of an electron in the conjugated polymer doped with hydrogen gas. By controlling |ΔG| to 4.5 eV or more, the interaction between the conjugated polymer and the dopant (A) can be reduced, so that the attraction of an electron from the conjugated polymer by the dopant (A) can be reduced. Accordingly, the positive charge of the conjugated polymer doped with the dopant (A) decreases. As a result, with a |ΔG| of 4.5 eV or more, the sensitivity of the hydrogen sensor element can be enhanced.

From the viewpoint of improving the sensitivity of the hydrogen sensor element, |ΔG| is preferably 4.6 eV or more, more preferably 4.7 eV or more, and still more preferably 4.8 eV or more.

|ΔG| is usually 10 eV or less, and preferably 8 eV or less from the viewpoint of easily causing the interaction between the conjugated polymer and the dopant (A).

Although the hydrogen detection film 103 may further contain a dopant other than the dopant (A) together with the dopant (A), it is preferable that only the dopant (A) be contained.

The LUMO energy of the dopant and the HOMO energy of the conjugated polymer may be determined from a DFT (Density Functional Theory; APFD/6-31G+g (d)) calculation based on the molecular structures, using a generalized calculation software. Examples of the calculation software include a quantum chemistry calculation program “Gaussian series” manufactured by HULINKS.

It is preferable that the dopant (A) have a high boiling point from the viewpoint of suppressing reduction in sensitivity of the hydrogen sensor element through suppression of desorption from the conjugated polymer. The boiling point of the dopant under atmospheric pressure is preferably 80° C. or more, more preferably 100° C. or more, and still more preferably 130° C. or more.

In the case where the hydrogen detection film 103 contains two or more types of dopants (A), it is preferable that at least one has a boiling point in the range, and it is more preferable that all of the dopants (A) have a boiling point in the range.

The dopant contained in the hydrogen detection film 103 is preferably an organic dopant. The use of an organic dopant is advantageous in controlling the doping percentage to an appropriate value. In the case of using an inorganic dopant such as inorganic acid having a high acidity function, the doping percentage increases too high to easily cause oxidative decomposition of the conjugated polymer.

Incidentally, in the case of using a polymer dopant as organic dopant, the polymer tends to embrace moisture, so that the influence of humidity on the electric resistance value detected by the hydrogen detection film 103 increases, easily resulting in reduction in the reliability of the hydrogen sensor element.

Examples of the dopant (A) include a compound that functions as an acceptor for the conjugated polymer.

As the acceptor dopant (A), in the case of the conjugated polymer of polyaniline-based polymer, an organic acid such as an organic carboxylic acid, an organic sulfonic acid, and an organic phosphonic acid, and a phosphoric acid ester are preferably used, and an organic sulfonic acid and an organic phosphonic acid are more preferably used. In the case of the conjugated polymer of polyaniline-based polymer, an organic acid, a phosphate esters, etc. have low proton donating properties, so that the polyaniline-based polymer is hardly oxidatively decomposed. As a result, the long-term stability of the hydrogen detection film 103 tends to be improved. Incidentally, the dopant (A) may be an inorganic acid such as hydrochloric acid.

Examples of the organic acid include ethanesulfonic acid, hydroxypropanesulfonic acid, 3-amino-1-propanesulfonic acid, aminoethylsulfonic acid, malonic acid, succinic acid, 1,5-pentylene diphosphonic acid, p-xylylene diphosphonic acid, dodecylphosphonic acid, and octadecylphosphonic acid.

Examples of the phosphoric acid ester include bis(2-ethylhexyl)phosphate, dibutyl phosphate, didecyl phosphate, diphenyl phosphate, and dibenzyl phosphate.

A preferred example of the hydrogen detection film 103 has a form in which the conjugated polymer is a polyaniline-based polymer and the dopant (A) is an organic dopant.

Another preferred example of the hydrogen detection film 103 has a form in which the conjugated polymer is a polyaniline-based polymer and the dopant (A) is an organic acid.

It is preferable that the dopant (A) contained in the hydrogen detection film 103 be an organic dopant having a molecular volume of 0.20 nm³ or less, or 0.25 nm³ or more. Thereby, the reversibility of the electric resistance value of the hydrogen sensor element can be improved.

In the case where the hydrogen concentration changes in a target (for example, an environment) for the measurement of hydrogen concentration with the hydrogen sensor element, the reversibility of the electric resistance value referred to here means the ability capable of having the same sensitivity in the case of the same change in the hydrogen concentration. For example, in the case where the hydrogen concentration in a measurement target changes from A to B to A to B, the sensitivity for the first change in hydrogen concentration from A to B is the same as the sensitivity for the second change in hydrogen concentration from A to B, or in the case where the difference between them is small, it can be said that the hydrogen sensor element has good reversibility.

The sensitivity referred to here is a percentage change in the electric resistance value indicated by the hydrogen sensor element.

One of the reasons why the reversibility of the electric resistance value of the hydrogen sensor element is improved with a molecular volume of the dopant (A) of 0.20 nm³ or less is presumed that the dopant easily allows the hydrogen molecule approaches or leaves the doping site of the conjugated polymer.

Further, with a molecular volume of the organic dopant of 0.20 nm³ or less, it is presumed that hydrogen gas easily penetrates the hydrogen detection film 103, which is presumed to be advantageous for improving the sensitivity of the hydrogen sensor element.

It is preferable that the dopant (A) having a molecular volume of 0.20 nm³ or less contain no fluorine atom from the viewpoint of improving the sensitivity of the hydrogen sensor element.

One of the reasons why the reversibility of the electric resistance value of the hydrogen sensor element is improved with a molecular volume of the dopant (A) of 0.25 nm³ or more is presumed as follows.

That is, with a molecular volume of the organic dopant of 0.25 nm³ or more, the organic dopant hardly penetrates deep into the hydrogen detection film 103 to be doped due to the steric hindrance of the organic dopant, being doped on or near the surface of the hydrogen detection film 103. As a result, the hydrogen gas is also doped/dedoped on or near the surface of the hydrogen detection film 103, so that the doping/dedoping is easily performed and the reversibility of the electric resistance value is improved.

Further, with a molecular volume of the organic dopant of 0.25 nm³ or more, it is presumed that desorption from the conjugated polymer hardly occurs due to the structure or steric hindrance of the organic dopant, which is presumed to be advantageous for the improvement in long-term stability of the hydrogen sensor element.

In the case where the molecular volume of the dopant (A) is 0.20 nm³ or less, from the viewpoint of improving the reversibility of the electric resistance value, the molecular volume of the dopant (A) is preferably 0.18 nm³ or less, more preferably 0.16 nm³ or less, and still more preferably 0.15 nm³ or less.

The molecular volume of the dopant (A) is usually 0.05 nm³ or more, and preferably 0.06 nm³ or more from the viewpoint of improving the long-term stability of the hydrogen detection film 103.

In the case where the molecular volume of the dopant (A) is 0.25 nm³ or more, from the viewpoint of improving the reversibility of the electric resistance value, the molecular volume of the dopant (A) is preferably 0.27 nm³ or more, more preferably 0.29 nm³ or more, and still more preferably 0.30 nm³ or more.

The molecular volume of the dopant (A) is usually 0.60 nm³ or less, and from the viewpoint of appropriately increasing the doping percentage through increase in the easiness of penetration into the conjugated polymer, preferably 0.50 nm³ or less, more preferably 0.45 nm³ or less.

The molecular volume of an organic dopant changes depending on the sizes, the steric structure, etc. of the atoms constituting the dopant.

The molecular volume of the dopant may be determined from a DFT (Density Functional Theory; B3LYP/6-31G+g (d)) calculation based on the molecular structures, using a generalized calculation software. Examples of the calculation software include a quantum chemistry calculation program “Gaussian series” manufactured by HULINKS.

Examples of the combination of the conjugated polymer having |ΔG| of 4.5 eV or more and the dopant (A) having a molecular volume of 0.20 nm³ or less include polyaniline and ethanesulfonic acid, polyaniline and hydroxypropanesulfonic acid, and polyaniline and 3-amino-1-propanesulfonic acid, polyaniline and aminoethylsulfonic acid, polyaniline and malonic acid, and polyaniline and succinic acid.

Examples of the combination of the conjugated polymer having |ΔG| of 4.5 eV or more and the dopant (A) having a molecular volume of 0.25 nm³ or more include polyaniline and diphenyl phosphate, polyaniline and bis(2-ethylhexyl)phosphate, polyaniline and dibutyl phosphate, polyaniline and didecyl phosphate, polyaniline and dibenzyl phosphate, polyaniline and p-xylylene diphosphonic acid, polyaniline and dodecylphosphonic acid, and polyaniline and octadecylphosphonic acid.

It is preferable that the dopant (A) contained in the hydrogen detection film 103 be an organic dopant having a dipole moment of 6 D (Debye) or less. Thereby, the humidity dependence of the electric resistance value indicated by the hydrogen sensor element can be reduced (the electric resistance value can be less affected by the humidity of the measurement environment), so that the function and/or reliability of the hydrogen sensor element can be further improved.

The reason why the humidity dependence of the electric resistance value can be reduced with a dipole moment of the dopant (A) of 6 D or less is presumed that the dopant hardly attracts water, due to having a low affinity with water that is a polar molecule.

From the viewpoint of reducing the humidity dependence of the electric resistance value, the dipole moment of the dopant (A) is preferably 5 D or less, more preferably 4.5 D or less, still more preferably 4 D or less, and particularly preferably 3.5 D or less.

The dipole moment of the dopant (A) is usually 0.1 D or more, preferably 0.5 D or more, and more preferably 1 D or more from the viewpoint of compatibility with the conjugated polymer.

The dipole moment of an organic dopant changes depending on the electronegativity and the steric structure of the atoms that compose the dopant.

The dipole moment of the organic dopant may be determined from a DFT (Density Functional Theory; B3LYP/6-31G+g (d)) calculation based on the molecular structures, using a generalized calculation software. Examples of the calculation software include a quantum chemistry calculation program “Gaussian series” manufactured by HULINKS.

Examples of the combination of the conjugated polymer having |ΔG| of 4.5 eV or more and the dopant (A) having a dipole moment of 6 D or less include polyaniline and 3-amino-1-propanesulfonic acid, polyaniline and aminoethylsulphonic acid, polyaniline and hydroxypropanesulfonic acid, polyaniline and ethanesulfonic acid, polyaniline and bis(2-ethylhexyl)phosphate, polyaniline and dibutyl phosphate, polyaniline and didecyl phosphate, polyaniline and diphenyl phosphate, polyaniline and dibenzyl phosphate, polyaniline and butylphosphonic acid, polyaniline and 1,5-pentylene diphosphonic acid, polyaniline and p-xylylene diphosphonic acid, polyaniline and dodecylphosphonic acid, polyaniline and octadecylphosphonic acid, polyaniline and malonic acid, and polyaniline and succinic acid.

From the viewpoint of further reducing the humidity dependence of the electric resistance value, it is more preferable that the dopant (A) satisfy any one or more of the following in addition to having a dipole moment of 6 D or less.

(a) Having a hydrophobic group such as alkyl group in the molecule.

(b) Having at least one, preferably two or more aromatic rings (for example, a benzene ring) in the molecule, for example, in the case where the conjugated polymer has an aromatic ring (for example, a benzene ring) such as polyaniline-based polymer.

(c) Having a fluorine atom in the molecule.

By satisfying the above (a), the water insolubility of the dopant (A) can be increased, so that the humidity dependence can be further reduced. However, rather than being water insoluble, having a small degree of uneven distribution of electric charges due to small dipole moment tends to hardly attract water.

The reason why the humidity dependence can be further reduced by satisfying the above (b) is presumed that the packing property of the dopant (A) with the conjugated polymer is improved.

In addition to the above, the molecular structure of the organic dopant and the type of functional group that the organic dopant has may affect the humidity dependence. For example, having a hydrophilic group tends to increase the humidity dependence.

It is preferable that the dopant (A) contained in the hydrogen detection film 103 be an organic dopant having an acid group, containing an atom having an absolute value of negative charge of 0.55 or more (hereinafter, the atom is also referred to as “atom a”) in the molecular structure other than the acid group. Thereby, the sensitivity of the hydrogen sensor element can be improved. As the atom a, among the atoms contained in the molecular structure other than the acid group, an atom having the largest absolute value of the negative charge is usually selected.

In order to increase the sensitivity (reactivity to hydrogen) of the hydrogen sensor element, it is important to reduce the positive charge of the conjugated polymer. Reducing the positive charge in the conjugated polymer means reducing the attraction of an electron from the conjugated polymer by the dopant, which leaves room for attraction of an electron in the conjugated polymer doped with hydrogen gas. In the dopant (A) containing the atom a in the molecular structure other than the acid group, the charge of the atom around the atom a is positively large, and accordingly the positive charge of the acid group is small. Thereby, the dopant (A) has a weaker force to pull out an electron from the conjugated polymer, and therefore the positive charge of the conjugated polymer doped with the dopant (A) is reduced. As a result, the sensitivity of the hydrogen sensor element can be increased by containing the dopant (A) containing the atom a in the molecular structure other than the acid group.

From the viewpoint of improving the sensitivity of the hydrogen sensor element, the absolute value of the negative charge of the atom a is preferably 0.6 or more, more preferably 0.65 or more.

The absolute value of the negative charge of the atom a is usually 1.5 or less, and preferably 1.2 or less from the viewpoint of imparting a function as acceptor.

The charge of the dopant may be determined from a DFT (Density Functional Theory; APFD/6-31G+g (d)) calculation based on the molecular structures, using a generalized calculation software, and subsequent optimization of the charge by the MK method of electrostatic potential fitting (esp). Examples of the calculation software include a quantum chemistry calculation program “Gaussian series” manufactured by HULINKS.

Examples of the combination of the conjugated polymer having |ΔG| of 4.5 eV or more and the dopant (A) having a dipole moment of 6 D or less, where the dopant (A) is an organic dopant having an acid group and containing the atom a in the molecular structure other than the acid group, include polyaniline and hydroxypropanesulfonic acid, and polyaniline and 3-amino-1-propanesulfonic acid, and polyaniline and aminoethylsulfonic acid.

The content of the dopant (A) is preferably 0.1 mol or more, more preferably 0.4 mol or more, relative to 1 mol of the conjugated polymer, from the viewpoint of increasing the sensitivity of the hydrogen sensor element. The content is preferably 3 mol or less, more preferably 2 mol or less, relative to 1 mol of the conjugated polymer, from the viewpoint of film formability in forming of the hydrogen detection film 103.

[3-3] Thickness of Hydrogen Detection Film

The thickness of the hydrogen detection film 103 is not particularly limited, being, for example, 0.3 μm or more and 50 μm or less. From the viewpoint of the flexibility of the hydrogen sensor element, the thickness of the hydrogen detection film 103 is preferably 0.3 or more and 40 μm or less.

[4] Hydrogen Sensor Element

The hydrogen sensor element may be manufactured, for example, by preparing a substrate 104 having a pair of electrodes composed of a first electrode 101 and a second electrode 102, and forming the hydrogen detection film 103 in contact with both the first electrode 101 and the second electrode 102.

The hydrogen detection film 103 may be manufactured, for example, by forming a film (layer) of conjugated polymer through a polymerization reaction on a substrate 104, and then impregnating the film with a dopant.

Examples of the polymerization reaction on the substrate 104 include a method including disposing a liquid containing a monomer to form the conjugated polymer and a liquid containing a polymerization initiator on the substrate 104 in a superposed manner. The substrate may be heated to accelerate the polymerization reaction on an as needed basis.

The hydrogen sensor element may include other components other than those described above. Examples of the other components include an antioxidant, metal fine particles, metal oxide fine particles, and graphite.

The antioxidant may contribute the prevention of oxidation of the hydrogen detection film 103. The metal fine particles, metal oxide fine particles, and graphite may contribute to improving the sensitivity of the hydrogen sensor element.

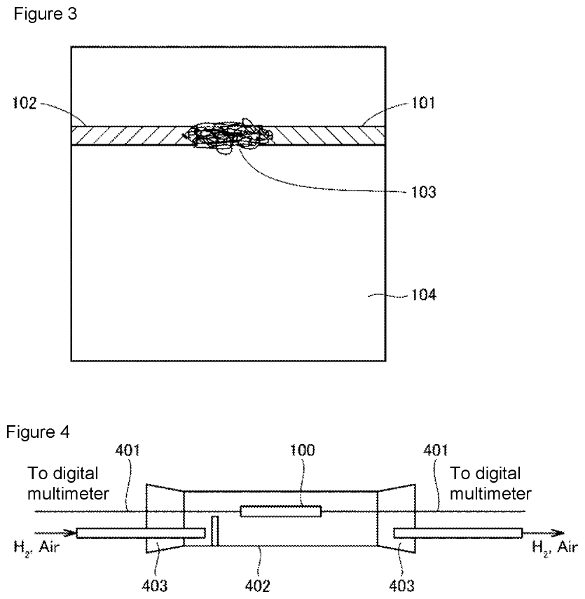

The hydrogen sensor element according to the present invention has good sensitivity to changes in the hydrogen concentration of a measurement target. The sensitivity of the hydrogen sensor element may be evaluated by a percentage change in the electric resistance value indicated by the hydrogen sensor element when the hydrogen concentration of the measurement target changes, and may be evaluated for example, by the following method. First, as shown in FIG. 2, a pair of electrodes (first electrode 101 and second electrode 102) made of Au is formed on one surface of a glass substrate (substrate 104), and then as shown in FIG. 3, a hydrogen detection film 103 is formed in contact with both of these electrodes, so that a hydrogen sensor element is manufactured.

Next, with reference to FIG. 4, the pair of Au electrodes of the hydrogen sensor element 100 and a commercially available digital multimeter are connected with a lead wire 401, and the hydrogen sensor element 100 is housed in a cylindrical container 402. Then, both ends of the container are sealed with a rubber stopper 403 provided with an outlet of the lead wire 401 and a gas inlet/outlet.

While monitoring the electric resistance value with a digital multimeter, a test is conducted in which gas is flowed into the container 402 in the order of the following [1] to [2] for the following time. The following test is performed in an environment at a temperature of 23° C.

[1] Dry air only at a flow rate of 10 L/min (hydrogen concentration: 0 vol %) for 10 minutes

[2] Dry air at a flow rate of 10 L/min and dry air (mixed gas having a hydrogen concentration of 2 vol %) at a flow rate of 0.2 L/min for 10 minutes

Then, a percentage change in electrical resistance value Z (%) is determined based on the following formula. The percentage change in electric resistance value Z may be used as an index (sensitivity index) representing the sensitivity of the hydrogen sensor element.

$$Percentage change in electric resistance value Z = \qquad [ Numerical Formula 1 ]$$

$$\frac{\begin{matrix}\vert Electric resistance value H 2 - \\ Electric resistance value H 0 \vert\end{matrix}}{Electric resistance value H 0} \times 100$$

In the formula, the electric resistance value H2 is an average value of the electric resistance values from 3 minutes to 10 minutes after switching the gas introduced into the container 402 to a mixed gas having a hydrogen concentration of 2 vol %. The electric resistance value HO is an average value of the electric resistance values from 3 minutes to 10 minutes after introducing only the dry air into the container 402.

From the viewpoint of enhancing the function and/or reliability as a hydrogen sensor element, it is preferable that the percentage change in electric resistance value Z be high. The electrical resistance value change rate Z may be, for example, 1% or more at 23° C., preferably 4% or more, more preferably 5% or more, still more preferably 6% or more, furthermore preferably 7% or more, and particularly preferably 8% or more. The percentage change in electrical resistance value Z may be 25% or less at 23° C.

According to the present invention, a hydrogen sensor element having good sensitivity in various purposes and usage environments can be provided.

The humidity dependence of the electric resistance value of a hydrogen sensor element may be evaluated, for example, by the following method.

After manufacturing a hydrogen sensor element, the hydrogen sensor element is exposed to dry air overnight, and a pair of Au electrodes of the hydrogen sensor element and a commercially available digital multimeter are connected with a lead wire.

Subsequently, while monitoring the electric resistance value with a digital multimeter, the hydrogen sensor element is allowed to stand in an atmosphere at a temperature of 30° C. and a relative humidity of 30% RH for 30 minutes. Subsequently, while monitoring the electric resistance value with a digital multimeter, the hydrogen sensor element is allowed to stand in an atmosphere at a temperature of 30° C. and a relative humidity of 80% RH for 30 minutes. From the electric resistance values under the respective atmospheres, the humidity dependence index value (%) of the electric resistance value is determined based on the following formula.

$$Humidity dependence index value = \qquad [ Numerical Formula 2 ]$$

$$\frac{\begin{matrix}\vert Electric resistance value RH 80 - \\ Electric resistance value RH 30 \vert\end{matrix}}{Electric resistance value RH 30} \times 100$$

In the formula, the electric resistance value RH30 is an electric resistance value when left standing in an atmosphere having a temperature of 30° C. and a relative humidity of 30% RH, more specifically, an average value of the electric resistance values from a standing time of 15 minutes to 30 minutes. The electric resistance value RH80 is an electric resistance value when left standing in an atmosphere having a temperature of 30° C. and a relative humidity of 80% RH, more specifically, an average value of electric resistance values from a standing time of 15 minutes to 30 minutes.

The smaller the difference between the electric resistance value RH80 and the electric resistance value RH30, the smaller the index value of humidity dependence. Therefore, it can be said that the smaller the index value of humidity dependence, the smaller the humidity dependence of the hydrogen sensor element.

The index value of humidity dependence is preferably less than 30%, more preferably 25% or less, still more preferably 20% or less, furthermore preferably 15% or less, and particularly preferably 10% or less. The index value of humidity dependence may be 1% or more.

### EXAMPLES

Hereinafter, the present invention will be described in more detail with reference to Examples, though the present invention is not limited thereto. In Examples, % and parts representing the content or the amount used are based on mass unless otherwise specified.

### Example 1

With reference to FIG. 2, on one surface of a square glass substrate (“Eagle XG” manufactured by Corning Inc.) having a side of 5 cm, a pair of rectangular Au electrodes having a length of 2 cm and a width of 3 mm was formed by sputtering using an ion coater (“IB-3” manufactured by Eiko Corporation).

The thickness of the Au electrodes determined by cross-sectional observation using a scanning electron microscope (SEM) was 200 nm.

A solution A containing 0.029 g of ammonium persulfate (manufactured by Fuji Film Wako Pure Chemical Corporation) dissolved in 1.55 mL of 1 M hydrochloric acid, and a solution B containing 0.48 g of aniline (manufactured by Tokyo Chemical Industry Co., Ltd.) dissolved in 1.2 mL of xylene (manufactured by Tokyo Chemical Industry Co., Ltd.) were prepared.

Between the pair of Au electrodes formed on the glass substrate, 90 μL of the solution A was dropped, and 10 μL of the solution B was further dropped thereto. The mixture was left standing for 5 minutes to perform a polymerization reaction.

Then, the glass substrate was moved to a spin coater (“MS-A100” manufactured by Mikasa) and rotated under a condition at 3000 rpm/30 s to remove the polymerization field from the glass substrate, so that a film of polyaniline emeraldine salt represented by the following formula (1) was obtained. After drying at room temperature for 20 minutes, the film was immersed in a two-fold diluted 25% aqueous ammonia (manufactured by Fuji Film Wako Pure Chemical Corporation). When the film color was changed from green to blue due to dedoping of polyaniline, the film was taken out from the aqueous ammonia and washed with water.

Then, the film was dried to obtain a nanofiber film of a polyaniline emeraldine base in a dedoped state represented by the following formula (2). The formed film was in contact with both electrodes (FIG. 3).

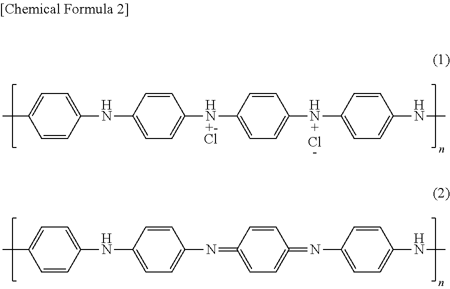

Subsequently, in a dopant solution 1 containing 1 g of hydroxypropanesulfonic acid (manufactured by Tokyo Kasei Kogyo Co., Ltd.) dissolved in 19 g of distilled water, the dedoped polyaniline nanofiber film formed on a glass substrate was immersed at a temperature of 23° C., and left standing for 2 hours to be redoped. Then, the film (hydrogen detection film) was dried with dry air for 12 hours to obtain a hydrogen sensor element. The thickness of the hydrogen detection film measured with Dektak KXT (manufactured by BRUKER) was 30 μm.

### Example 2

A hydrogen sensor element was manufactured in the same manner as in Example 1, except that a dopant solution 2 containing 1 g of ethanesulfonic acid (manufactured by Tokyo Kasei Kogyo Co., Ltd.) dissolved in 19 g of distilled water was used instead of the dopant solution 1 as the dopant solution for immersion of the polyaniline nanofiber film. The thickness of the hydrogen detection film measured in the same manner as in Example 1 was 30 μm.

### Example 3

A hydrogen sensor element was manufactured in the same manner as in Example 1, except that a dopant solution 3 containing 1 g of 1,5-pentylene disulfonic acid (manufactured by Tokyo Kasei Kogyo Co., Ltd.) dissolved in 19 g of distilled water was used instead of the dopant solution 1 as the dopant solution for immersion of the polyaniline nanofiber film. The thickness of the hydrogen detection film measured in the same manner as in Example 1 was 30 μm.

### Comparative Example 1

A hydrogen sensor element was manufactured in the same manner as in Example 1, except that a dopant solution 4 containing 1 g of camphorsulfonic acid (manufactured by Tokyo Kasei Kogyo Co., Ltd.) dissolved in 19 g of distilled water was used instead of the dopant solution 1 as the dopant solution for immersion of the polyaniline nanofiber film. The thickness of the hydrogen detection film measured in the same manner as in Example 1 was 30 μm.

The types of dopants used in Examples and Comparative Examples, and the absolute value |ΔG| of the energy difference between the lowest unoccupied orbital (LUMO) of the dopant and the highest occupied orbital (HOMO) of the conjugated polymer are shown in Table 1.

The LUMO energy of the dopant and the HOMO energy of the conjugated polymer was determined from a DFT (Density Functional Theory; APFD/6-31G+g (d)) calculation based on the molecular structures, using a quantum chemistry calculation program “Gaussian 16” manufactured by HULINKS.

[Evaluation of Hydrogen Sensor Element]

The sensitivity of the hydrogen sensor elements obtained in Examples and Comparative Examples was evaluated by the following test.

With reference to FIG. 4, the manufactured hydrogen sensor element was exposed to dry air overnight, and the pair of Au electrodes of the hydrogen sensor element 100 and a digital multimeter (“XDM3051” manufactured by OWON) were connected with a lead wire 401. The hydrogen sensor element 100 was then housed in an acrylic cylinder (container 402). Then, both ends of the container were sealed with a rubber stopper 403 provided with an outlet of the lead wire 401 and a gas inlet/outlet.

While monitoring the electric resistance value with a digital multimeter, a test was conducted in which gas was flowed into the container 402 in the order of the following [1] to [2] for the following time. The following test was performed in an environment at a temperature of 23° C.

[1] Dry air only at a flow rate of 10 L/min (hydrogen concentration: 0 vol %) for 10 minutes

[2] Dry air at a flow rate of 10 L/min and dry air (mixed gas having a hydrogen concentration of 2 vol %) at a flow rate of 0.2 L/min for 10 minutes

Based on the above test results, the percentage change in electrical resistance Z as defined above was determined following the formula described above. The percentage change in electric resistance value Z (sensitivity index) is shown in Table 1.

TABLE 1

| | | | Percentage |
|---|---|---|---|
| | | | change in electric |
| | | | resistance value |
| | | \| Δ G \| | Z (Sensitivity |
| | Dopant | (eV) | index) (%) |
| Example 1 | Hydroxypropanesulfonic | 4.87 | 10.7 |
| | acid | | |
| Example 2 | Ethanesulfonic acid | 4.90 | 8.7 |
| Example 3 | 1,5-Pentylene disulfonic | 4.82 | 8.8 |
| | acid | | |
| Comparative | Camphorsulfonic acid | 4.22 | 3.7 |
| Example 1 | | | |

1. A hydrogen sensor element comprising a pair of electrodes and a hydrogen detection film disposed in contact with the pair of electrodes,
wherein the hydrogen detection film contains a conjugated polymer and a dopant, and
wherein an absolute value |ΔG| of energy difference between the lowest unoccupied orbital of the dopant and the highest occupied orbital of the conjugated polymer in the ground state is 4.5 eV or more.

2. The hydrogen sensor element according to claim 1, wherein the conjugated polymer is a polyaniline-based polymer.

3. The hydrogen sensor element according to claim 1, wherein the dopant is an organic dopant.

4. The hydrogen sensor element according to claim 1, wherein the hydrogen detection film contains nanofibers of the conjugated polymer.

5. The hydrogen sensor element according to claim 3, wherein the organic dopant has a dipole moment of 6 D or less.

6. The hydrogen sensor element according to claim 3, wherein the organic dopant has a molecular volume of 0.20 nm³ or less.

7. The hydrogen sensor element according to claim 1, wherein the dopant has a boiling point of 80° C. or more under atmospheric pressure.

8. The hydrogen sensor element according to claim 1, wherein the dopant is an organic sulfonic acid.
